(19) United States
(12) Plant Patent Application Publication
(10) Pub. No.: US 2003/0177554 P1
(43) Pub. Date: Sep. 18, 2003

(54) Spray rose plant named 'meicandy'

(76) Inventor: Alain A. Meilland, Antibes (FR)

Correspondence Address: BURNS, DOANE, SWECKER & MATHIS, L.L.P., P.O. Box 1404, Alexandria, VA, 22313-1404, US

(21) Appl. No.: 10/387,548

(22) Filed: Mar. 14, 2003

Related U.S. Application Data
(63) Continuation of application No. 09/648,651, filed on Aug. 28, 2000.

(51) Int. Cl. A01H 5/00

(52) U.S. Cl. PLT/142

(57) ABSTRACT

A new and distinct variety of rose plant of the Spray Class is provided which abundantly forms attractive double bicolored blossoms that are Currant Red on the upper surface and Orient Pink on the under surface. The buds are conical in configuration and are borne on relatively long stems that bear few if any thorns. An erect growth habit is exhibited. The foliage is dense and medium green and contrasts nicely with the red and pink blossoms. The plant is well suited for the production of cut floral sprays under greenhouse growing conditions.

### BOTANICAL/COMMERCIAL CLASSIFICATION

Rose hybrida/Spray Rose

### VARIETAL DENOMINATION

cv. ‘Meicandy’

### SUMMARY OF THE INVENTION

The new variety of Rosa hybrida spray rose plant was created by artificial pollination wherein two parents were crossed which previously had been studied in the hope that they would contribute the desired characteristics. The female parent (i.e., the seed parent) of the new variety was the ‘Korokis’ variety (non-patented in the United States). The male parent (i.e., the pollen parent) of the new variety was the ‘Olytel’ variety (U.S. Plant Pat. No. 8,862). The parentage of the new variety can be summarized as follows:

‘Korokis’×‘Olytel’.

The seeds resulting from the above pollination were sown and small plants were obtained which were physically and biologically different from each other. Selective study resulted in the identification of a single plant of the new variety.

It was found that the new variety of spray rose plant of the present invention possesses the following combination of characteristics:

(a) forms conical buds in the form of a spray on relatively long stems having few thorns on the flowering stems,

(b) forms in abundance attractive bicolored blossoms that are Currant Red on the upper surface and Orient Pink on the under surface,

(c) exhibits an erect growth habit, and

(d) forms attractive semi-glossy medium green foliage.

The spray inflorescence coloration contrasts nicely with the dense medium green foliage. Cut flower rose plants that produce flowers in sprays can be designated as Floribunda rose plants.

The new variety of the present invention can be readily distinguished from its ancestors through a comparison of the blossom coloration. For instance, the ‘Korokis’ parent forms light pink blossoms and the ‘Olytel’ parent forms blossoms that display a pink blend coloration.

The new variety well meets the needs of the horticultural industry. It is particularly well-suited for use in the production of cut floral sprays under greenhouse growing conditions.

The new variety has been found to undergo asexual propagation in France by a number of routes, including budding, grafting, and cuttage. Asexual propagation by the above-mentioned methods as performed in France has shown that the characteristics of the new variety are strictly transmissible from one generation to another.

The new variety has been named the ‘Meicandy’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age were observed during January while growing on Rosa indica understock in greenhouses at LeCannet des Maures, Var, France.

FIG. 1—illustrates a specimen of a young shoot;

FIG. 2—illustrates a specimen of a group of three floral buds before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower during the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem wherein thorns are sparsely present;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface;

FIG. 16—illustrates a specimen of a leaf with seven leaflets—plan view—upper surface; and

FIG. 17—illustrates a specimen of a cluster of buds.

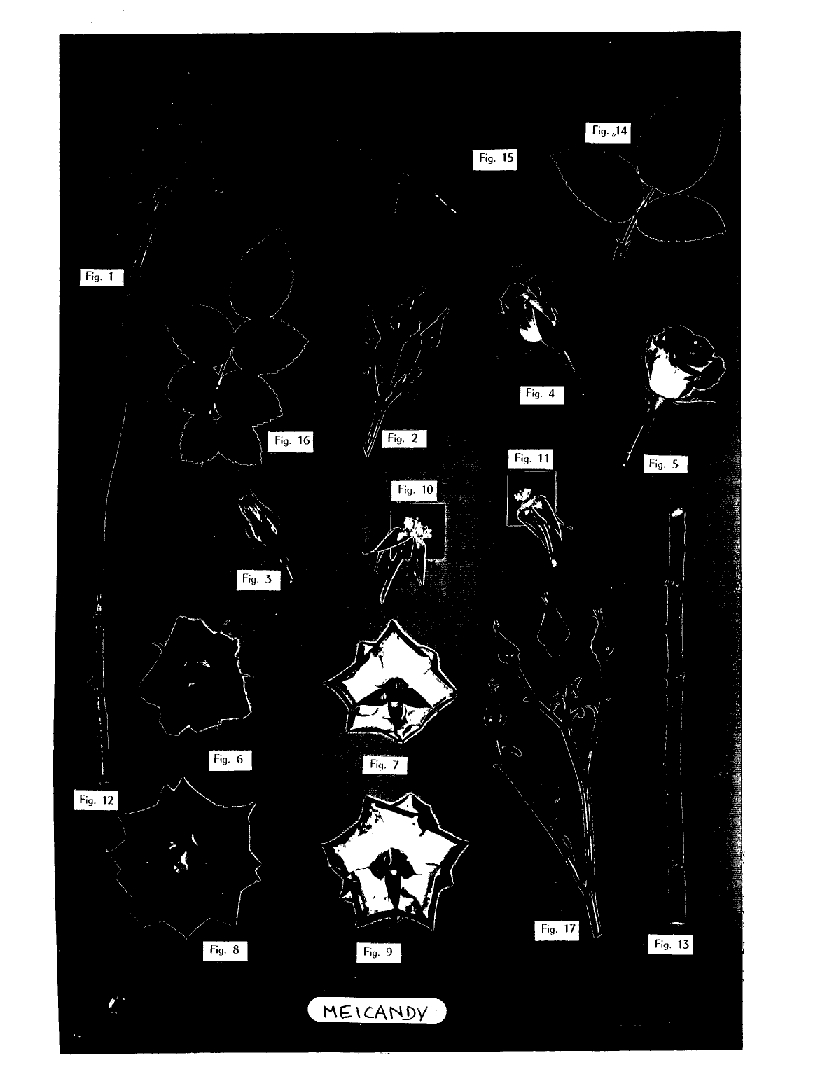

### DETAILED DESCRIPTION

The chart used in the identification of the colors is that of The Royal Horticultural Society (R.H.S. Colour Chart). When more common color terms are utilized they are to be accorded customary dictionary significance. The description is based on two year-old specimens of the new variety which were observed during July while budded on Rosa indica understock and growing in greenhouses at LeCannet des Maures, Var, France. Dimensions in centimeters are provided at the bottom of the photograph.

CLASS:

Spray.

PLANT:

Height.—a plant pruned to a height of 85 cm commonly produces floral stems having a relatively long length of approximately 50 to 70 cm for a rose plant of this Class.

Habit.—erect.

BRANCHES:

Color.—young stems: near Green Group 137C with some anthocyanin coloration of near Greyed-Red Group 178A. adult wood: near Green Group 137A.

Thorns.—size: generally small (as illustrated). quantity: few at most on flowering stems and moderately numerous on adult wood. Approximately 5 thorns on average are formed per 10 cm of adult stems. configuration: the upper surface is rather elongated and reflexed towards the base and the lower surface is concave. color: near Red Group 50A and 50B on young stems and near Greyed-Orange Group 17D on adult wood.

LEAVES:

Petioles.—slightly glandular and reddish anthocyanin coloration of near Greyed-Red Group 178A on the upper surface, and light green without prickles on the under surface.

Stipules.—adnate, pectinate, and narrow.

Leaflets.—number: 3, 5 (most often), and 7. shape: generally elliptic with a narrow generally symmetrical tip and a rounded base. texture: typical and normal. serration: regular. general appearance: rather dense, medium green, and semi-glossy. color (young foliage): upper surface: near Green Group 139A with some reddish anthocyanin coloration of near Greyed-Red Group 178A. under surface: Yellow-Green Group 147B with some reddish anthocyanin coloration. color (adult foliage): upper surface: near Yellow-Green Group 147A. under surface: near Yellow-Green Group 147B.

INFLORESCENCE

Number of flowers.—commonly approximately 6 to 15 per stem.

Peduncle.—rigid, near Yellow-Green Group 146C in coloration, without prickles, short, and the length is approximately 6 cm on average.

Sepals.—upper surface: tomentose, and near Yellow-Green Group 146B, 146C, and 146D in coloration. under surface: near Yellow-Green Group 146B in coloration. shape: with elongated tips and approximately 2 cm in length on average. Commonly three sepals have medium extensions and two sepals have no extensions.

Buds.—shape: conical. length: approximately 3 cm on average. size: small.

Flower.—form: high pointed. diameter: approximately 8 cm on average. color (when opening begins): upper surface: near Currant Red, Red Group 46A and very lightly suffused with Red Group 42A. under surface: near Orient Pink, Red Group 36D and widely suffused with Shell Pink, Red Group 37C and 37D. color (when blooming): upper surface: near Currant Red, Red Group 46A and very lightly suffused with Red Group 42A. under surface: near Orient Pink, Red Group 36D and widely suffused with Shell Pink, Red Group 37C and 37D. color (at end of opening): upper surface: near Currant Red, Red Group 46A and very lightly suffused with Red Group 42A. under surface: near Orient Pink, Red Group 36D and widely suffused with Shell Pink, Red Group 37C and 37D. fragrance: none. petal configuration: with a rounded base and a reflexed tip as illustrated. petal number: approximately 20 on average. petal drop: very good, the petals commonly detach cleanly. anthers: orange in coloration. pollen: formed in a moderate quantity and near Yellow Group 12C in coloration. stamen number: approximately 100 on average. filaments: near Yellow Group 8B in coloration. pistil number: approximately 35 on average. stigmas: near Yellow Group 8B in coloration. styles: near Yellow Group 8B in coloration. receptacle: near Yellow-Green Group 146B in coloration, and in longitudinal section in the shape of a pitcher. hips: substantially spherical in shape, possess a smooth surface, approximately 15 to 20 mm in diameter, and Yellow-Green Group 144A when observed on August 2nd. lasting quality: very long, approximately 12 to 14 days on the plant, and approximately 10 to 12 days when cut and placed in a vase.

DEVELOPMENT

Vegetation.—strong.

Blooming.—abundant.

Resistance to diseases.—excellent.

Aptitude to bear fruit.—good.

I claim:

1. A new and distinct variety of spray rose plant characterized by the following combination of characteristics:
(a) forms conical buds on relatively long stems having few thorns in the form of a spray,
(b) forms in abundance attractive bicolored blossoms that are Currant Red on the upper surface and Orient Pink on the under surface,
(c) exhibits an erect growth habit, and
(d) forms attractive semi-glossy medium green foliage;
substantially as herein shown and described.
